(19) United States
(12) Patent Application Publication
(10) Pub. No.: US 2024/0358541 A1
(43) Pub. Date: Oct. 31, 2024

(54) MEDICAL-DEVICE TENSIONING SYSTEMS, METHODS, AND KITS

(71) Applicant: Fistula Solution Corporation, Scandia, MN (US)

(72) Inventors: Andrew T. Obst, Scandia, MN (US); Mary Anne R. Obst, Scandia, MN (US)

(21) Appl. No.: 18/644,950

(22) Filed: Apr. 24, 2024

Related U.S. Application Data
(60) Provisional application No. 63/498,151, filed on Apr. 25, 2023.

(51) Int. Cl. A61F 5/449

(52) U.S. Cl. CPC A61F 5/449

(57) ABSTRACT

A tensioning system is configured to affix a medical device, such as an ostomy pouch, wound dressing, or medical tubing, to a user's body. In some examples, the tensioning system includes one or more elongated, adjustable flexible, stretchable straps to secure the medical device to the user's abdomen, increasing the tension of a flange of the medical device against the user's skin. A first end of each flexible strap is configured to removably couple to the medical device, e.g., via hooks, adhesive, laces, hook and loop, and/or a ring. A second end of each flexible strap (or a connector base thereupon) includes a skin-safe adhesive coating configured to adhere to the user's skin.

### RELATED APPLICATIONS

This application claims the benefit of U.S. Provisional Application Ser. No. 63/498,151, filed Apr. 25, 2023, which is incorporated herein by reference in its entirety.

### TECHNICAL FIELD

The present disclosure relates generally to medical devices and, more particularly, to

tensioning systems for securing an ostomy pouch, wound dressing, medical tube, or other medical device to a patient's body.

### BACKGROUND

Patients who have undergone surgery may be fitted with an ostomy pouch configured to collect waste diverted from the digestive or urinary tract through an opening (or “stoma”) on their abdomen. The ostomy pouch, which may be coupled to the patient's skin around the stoma, can be periodically disposed of and replaced.

One of the challenges of using an ostomy pouch is keeping it securely attached to the user's body. For instance, existing ostomy-pouch belts fitted with buckles, hooks, or other fasteners, can be difficult to adjust and may cause discomfort or irritation. Wearing belts too tightly can cause skin problems, e.g., pressure points or ulcers may develop under the hard plastic tabs, or irritation may occur where the tab rests on the user's skin. Some two-piece pouch systems can distort when belts are too tight, causing pouches to pop off their attachment flanges.

### SUMMARY OF THE DISCLOSURE

Embodiments of the present disclosure provides examples of an improved tensioning systems for securing ostomy pouches, wound dressings, medical tubes, and/or other medical devices to a user's body. In general, the medical-device tensioning system implements one or more adhesive straps to secure the device at its intended location (e.g., around the user's or subject's stoma). The adhesive straps are easily adjustable and can be removed and replaced as needed. The adhesive material of the straps is hypoallergenic and gentle on the skin, reducing the risk of irritation or discomfort.

In some examples, the tensioning system includes one or more flexible straps, each strap defining a first end and a second end. The first end of each strap is coupled to a medical device, the strap extends at partially around the user's body, and the second end is anchored to the user's skin via an adhesive. The medical device is coupled to the first end of each strap via a ring, washer, hooks, or disk that is disposed around or on the medical device. Alternatively, the medical device is coupled to the first end of each strap via an adhesive on the first end of the strap.

In some examples, the adhesive is a skin-compatible adhesive material coated onto one side of the strap, or on a flexible silicone or rubber strap connector base anchored to the skin. The straps are made of a flexible, stretchable material that conforms to the user's body and can be adjusted or repositioned on the body to provide a comfortable and secure fit.

In some examples, each strap is attached to the medical device with hooks, laces, hook and loop, or an adhesive. Additionally or alternatively, multiple straps can be connected to a ring disposed around the medical device, providing additional support and stability.

In an alternative example, the tensioning system can comprise a single flexible strap that can be adjusted and coupled to the subject. Each end of the strap includes the adhesive as described. The medical device can be coupled to the strap at a location between the ends, such as centered, or closer to one end than the other. Like the other embodiments, the medical device is coupled to the strap via a ring, washer, hook, or disk that is disposed around or on the medical device, or via adhesive.

In some examples, a tensioning system kit can comprise one or more flexible straps as described above, one or more medical devices, such as an ostomy bag, one or more coupling devices such as a optionally one or more adhesive pads, one or more coupling devices such as rings, washers, hooks, or disks, for coupling the medical device to the strap(s), and instructions for use. The instructions can include steps for positioning the tensioning system on a subject. For example, the instructions can provide steps such as providing a strap, coupling the strap to the medical device by coupling the coupling device to the medical device and the strap, removing a liner (if present) to expose the adhesive, positioning the adhesive on the skin, optionally repositioning the adhesive on the skin to reach a desired tension. In the event the adhesive is removable/replaceable, the instructions can further include replacing an adhesive pad position on the strap with a new adhesive pad in the kit for reuse of the strap.

The tensioning systems of this disclosure provide a number of benefits and advantages, including: improving ostomy-pouch adhesion, accentuating a convex ostomy-pouch system, and providing a sense of security for the user. For instance, the construction of the user's stoma (e.g., retraction or prolapse) and the characteristics of the user's skin around the stoma (e.g., flat, or any dips, creases, or folds) can diminish an ostomy pouch's fixation strength and thus, increase the risk of leakage. The tensioning systems described herein help secure the pouch to the user's abdomen, increasing the tension of the pouch flange against the user's skin. Additionally, the tension helps to flatten out skin folds, to re-orient the stoma so that it drains more effectively into the pouch, and to reduce or even prevent seepage of waste material underneath the flange.

The tensioning systems of this disclosure can also give users an enhanced sense of security. For those who actively participate in sports, enjoy soaks in baths or hot tubs, or readily perspire, the tensioning systems described herein offer improved support to alleviate concern regarding the adhesion of the pouch. Users can even wear the tensioning system while sleeping to prevent unwanted pouch movement, e.g., resulting from frequent sleep-position changes.

The details of one or more aspects of the disclosure are set forth in the accompanying drawings and the description below. Other features, objects, and advantages of the techniques described in this disclosure will be apparent from the description and the drawings, and from the claims.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a front view of a first example of a medical-device tensioning system, including adhesive straps coupled to an ostomy pouch via a pair of hooks.

FIG. 2 is a front view of a second example of the medical-device tensioning system of FIG. 1, including straps connected to adhesive skin anchors at one end, and attached to an ostomy pouch via adhesive at the other end.

FIG. 3 is a front view of a third example of the medical-device tensioning system of FIG. 1, including adhesive straps attached to a ring, washer, or disk that is disposed around the base of an ostomy pouch.

While examples of this disclosure are amenable to various modifications and alternative forms, specifics thereof shown by way of example in the drawings will be described in detail. It should be understood, however, that the intention is not to limit the disclosure to the particular examples described.

### DETAILED DESCRIPTION

The present disclosure describes various examples of devices, systems, and techniques for securing medical devices, such as ostomy pouches or medical tubing, to a user's body. In general, one or more adhesive straps are used to secure the medical device at its intended location (e.g., around a user's stoma). The adhesive straps are easily adjustable and can be repositioned, removed, and replaced as needed. The adhesive material of the straps is hypoallergenic and gentle on the skin, reducing the risk of irritation or discomfort.

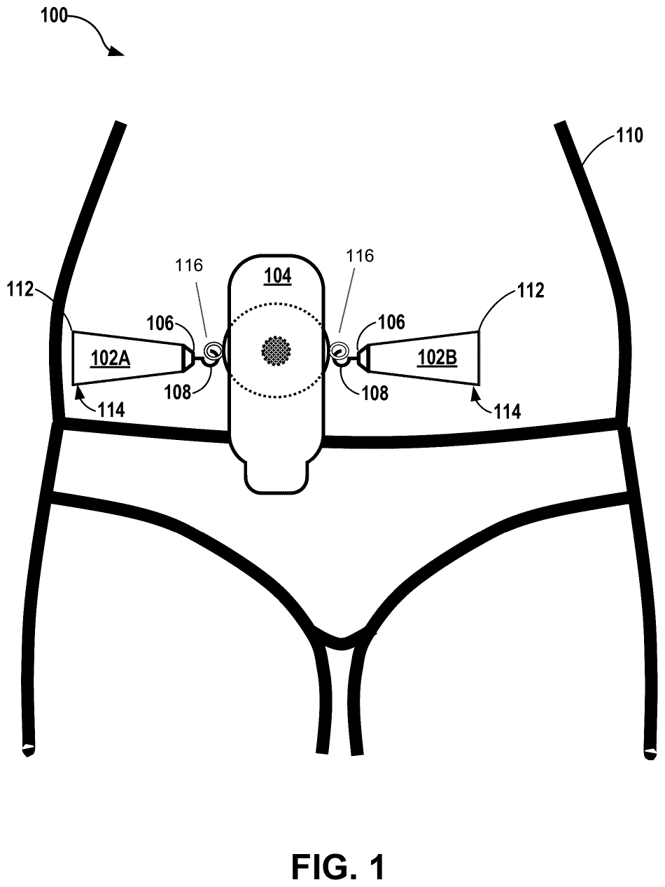

FIG. 1 shows a first example of a tensioning system 100 for securing and tensioning medical devices, such as ostomy pouches, medical tubes, and other medical devices, to a subject. In this example, system 100 can include two flexible straps 102A, 102B that are attached to an ostomy pouch 104. However, it is contemplated that more or less than two straps can be used. Each strap 102 has a first end 106 coupled to ostomy pouch 104 via a coupling device 108, such as a hook, that removably couples to a corresponding coupling device 116, such as a ring, coupled to ostomy pouch 104. Alternatively, hook 108 can be positioned on ostomy pouch 104 and ring 116 on strap 102. Middle portions of straps 102 extend at least partially around a user's body 110, wherein a second end 112 is secured to the user's skin via a skin-safe adhesive coating or pad 114, e.g., applied to an underside of straps 102. In some examples, adhesive 114 is generally positioned on second end 112, while in other examples, adhesive 114 covers a majority of the underside of strap 102. Optionally, a liner (not shown) covers adhesive 114, and is removed upon use. In yet another embodiment, strap 102 is impregnated with adhesive 114 such that a separate layer of adhesive is not present.

In example, straps 102 are made of a flexible, stretchable material that conforms to the user's body 110, and can be easily adjusted or repositioned on the body for a comfortable and secure fit. For example, straps 102 can be formed of a flexible elastic bandage material, a polymeric material, a composite material, and other examples, thereof, and can optionally provide compression.

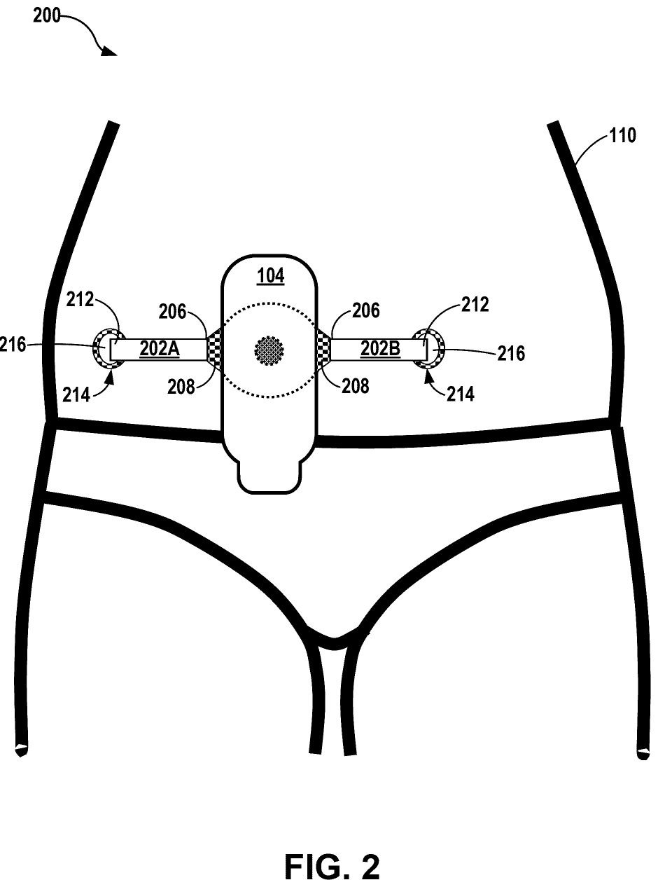

FIG. 2 shows another example medical-device tensioning system 200, which is an example of tensioning system 100 of FIG. 1. For instance, tensioning system 200 includes two flexible straps 202A, 202B (e.g., straps 102A, 102B of FIG. 1) coupled to ostomy pouch 104. Each strap 202 has a first end 206 attached to ostomy pouch 104 via adhesive 208. Each strap 202 extends at least partially around the user's body 110, where a second end 212 is secured to a flexible silicone or rubber base 216, which is anchored to the skin with a skin-safe adhesive coating 214. Straps 202 are made of a flexible, stretchable material that conforms to the user's body 110 and can be easily adjusted for a comfortable and secure fit, such as those listed with respect to system 100 supra.

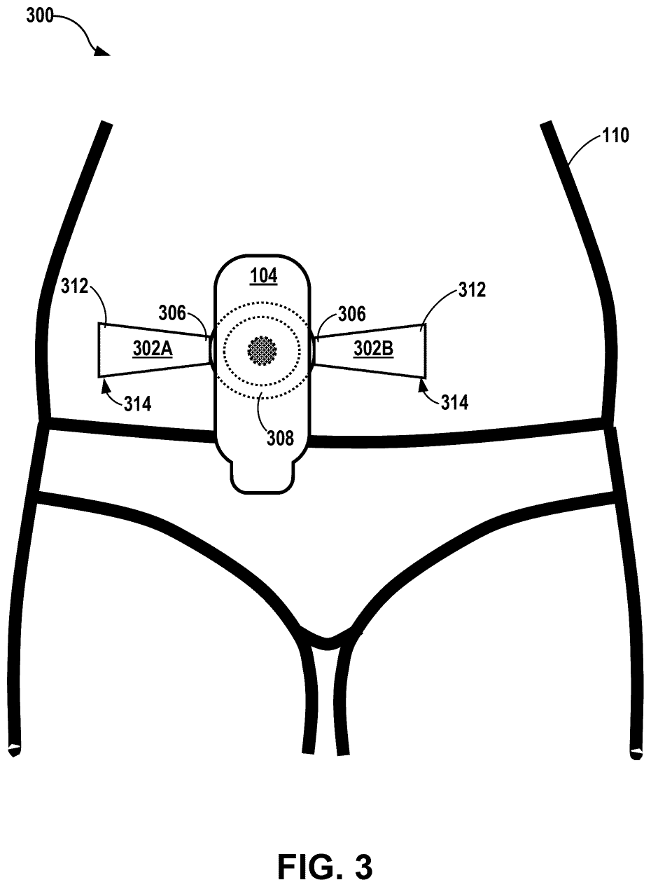

FIG. 3 shows another example medical-device tensioning system 300, which is an example of tensioning systems 100, 200 of FIGS. 1 and 2. For instance, tensioning system 300 includes two flexible straps 302A, 302B, each having a first end 306 coupled to a coupling device 308, such as a ring, washer, or disk, disposed around the base of ostomy pouch 104. First end 306 can be coupled to device 308 via looping, tying, adhesive, hook and ring, hook and loop, sewing, or any of a variety of attachment mechanisms. Middle portions of straps 302 extend at least partially around the user's body 110, wherein a second end 312 is secured to the user's skin via a skin-safe adhesive coating 314, e.g., applied to an underside of straps 302. Straps 302 are made of a flexible, stretchable material that conforms to the user's body 110 and can be easily adjusted or repositioned on the body for a comfortable and secure fit, as discussed supra. In some examples, the ring, washer, or disk 308 is selectively sizeable to fit snugly around ostomy pouch 104. Ostomy pouch 104 is then removably coupled to the coupling device 308.

In an alternative example, straps 302A, 302B and coupling device 308 are a single piece of unitary construction, rather than separate discrete pieces. In this embodiment, adhesive is coupled to each end of the unitary device, while coupling device 308 is formed along the unitary strap, such as in the center or offset from the center. In yet another embodiment, one or more straps 302 are directly coupled to ostomy pouch 104 and are not removably coupled thereto.

In examples, a tensioning system kit can comprise one or more flexible straps as described above, optionally one or more medical devices, such as an ostomy bag, optionally one or more adhesive pads, one or more coupling devices such as rings, washers, hooks, or disks, for coupling the medical device to the strap(s), and instructions for use. The instructions can include steps for positioning/repositioning/removing the tensioning system on a subject. For example, the instructions can provide steps such as providing one or more straps as described in the example above, optionally trimming the strap to a desired length, coupling the strap to the medical device by coupling the coupling device to the medical device and the strap (either before or after the straps are coupled to the subject), removing a liner (if present) to expose the adhesive, positioning the strap adhesive on the skin, optionally repositioning the adhesive on the skin to reach a desired tension, and/or removing the strap. In the event the adhesive is removable/replaceable, the instructions can further include replacing an adhesive pad position on the strap with a new adhesive pad in the kit for reuse of the strap.

In another example, the kit includes a roll of flexible material and adhesive pads either instead of or in addition to the straps described above. In this example, a strap can be prepared a user by cutting a desired length from the roll, applying an adhesive pad to a first end of the length, and applying a coupling device (hook, ring, adhesive, etc.) to the second end of the length. The strap is then utilized with the medical device as described supra.

The various examples of a medical-device tensioning system described herein provide several advantages over existing systems. For instance, multiple straps 102, 202, 302 and ring 308 provide additional support and stability to pouch 104, reducing the risk of detachment and/or leakage. The adhesive straps 102, 202, 302 are easy to adjust and can be repositioned, removed and replaced as needed, reducing discomfort and irritation. The adhesive material 114, 208, 214, 314 is also hypoallergenic and gentle on the skin. It is to be understood that the systems and techniques of this disclosure are not limited to the examples explicitly described with respect to FIGS. 1-3. On the contrary, the medical-device tensioning system is intended to cover all alternatives, modifications, and equivalents that may be included within the scope of the following claims.

Various examples of systems, devices, and methods have been described herein. These examples are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of this disclosure.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

What is claimed is:

1. A medical-device tensioning system for coupling a medical device to a subject, the system comprising:
at least one flexible, stretchable strap, each flexible strap including—
a first end configured to removably couple to the medical device,
a middle portion configured to extend at least partially around a body of a user; and
a second end configured to removably couple to a skin of the user.

2. The system of claim 1, wherein each flexible strap comprises an adjustment mechanism configured to modify a length of the flexible strap.

3. The system of claim 1, wherein an underside of each flexible strap comprises a skin-safe adhesive coating configured to couple the second end of the strap to the skin of the user and to be easily repositioned on the body for a comfortable and secure fit.

4. The system of claim 1, further comprising a flexible base coupled to the second end of each flexible strap, wherein the flexible base comprises a skin-safe adhesive coating configured to adhere to the skin of the user.

5. The system of claim 4, wherein the flexible base comprises silicone or rubber.

6. The system of claim 1, wherein the first end of each flexible strap comprises one or more hooks configured to removably couple to the medical device.

7. The system of claim 1, wherein the first end of each flexible strap comprises a cord, laces, or hook and loop configured to removably couple to the medical device.

8. The system of claim 1, wherein the first end of each strap comprises an adhesive portion configured to adhere to the medical device.

9. The system of claim 1, further comprising a ring, a washer, or a disk configured to be disposed around the medical device, wherein the first end of each flexible strap is configured to couple to the ring, the washer, or the disk.

10. The system of claim 9, wherein the ring, the washer, or the disk is selectively sizable.

11. The system of claim 1, wherein the medical device comprises an ostomy bag, wound dressing, or medical tubing.

12. The system of claim 9, wherein the ring, washer, or disk and the at least one flexible strap is a single piece of unitary construction.

13. A medical-device tensioning system for coupling a medical device to a subject, the system comprising:
a roll of flexible, stretchable material configured to be trimmed to a desired length comprising a first end, a middle portion, and a second end;
a coupling device for removably coupling the first end to the medical device; and
a source of adhesive configured to be coupled to the second end of the desired length such that the second end is configured to removably couple to a skin of the subject,
wherein the middle portion configured to extend at least partially around a body of the subject when the first end is coupled to the medical device and the second end is coupled to the skin of the subject.

14. The system of claim 13, wherein the coupling device comprises a hook configured to be coupled to the first end of the desired length.

15. The system of claim 13, wherein the coupling device comprises a cord, laces, or hook and loop, configured to be coupled to the first end of the desired length.

16. The system of claim 13, wherein the coupling device comprises an adhesive portion configured to be coupled to the first end of the desired length.

17. The system of claim 13, further comprising a ring, a washer, or a disk configured to be disposed around the medical device, wherein the first end of the desired length is configured to couple to the ring, the washer, or the disk.

18. A medical-device tensioning kit for coupling a medical device to a subject, the kit comprising:
a plurality of flexible, stretchable straps, each flexible strap including a first end configured to removably couple to the medical device, a middle portion configured to extend at least partially around a body of a user; and a second end configured to removably couple to a skin of the subject; and
a plurality of medical devices, each medical device being configured to couple to one or more straps of the plurality of straps.

19. The kit of claim 18, wherein the first end of each strap includes one or more hooks configured to removably couple to the medical device, a cord, laces, or hook and loop, configured to removably couple to the medical device, or an adhesive portion configured to adhere to the medical device,
wherein the second end of each strap includes an adhesive for removably securing the second end of the strap to the skin of the subject, and
wherein each medical device of the plurality of medical devices is an ostomy bag, wound dressing, medical tubing, or a combination thereof.

20. The kit of claim 18, further comprising instructions for use, the instructions for use including the steps of:
positioning the medical device on the subject;
securing the first end of a strap of the plurality of straps to the medical device;
positioning the second end of a strap of the plurality of straps to the skin of the subject; and
optionally repositioning the second end of the strap to the skin of the subject.
